(19) United States
(12) Patent Application Publication
(10) Pub. No.: US 2019/0046787 A1
(43) Pub. Date: Feb. 14, 2019

(54) WEARABLE CERVICAL TRANSDERMAL PULSED ELECTRICAL STIMULATION DEVICES AND CONTROL METHOD OF THE SAME

(71) Applicant: NEWDIN CO., LTD., Seoul (KR)

(72) Inventors: William J. Tyler, Cave Creek, AZ (US); Daniel Z. Wetmore, Brooklyn, NY (US)

(73) Assignee: NEWDIN CO., LTD., Seoul (KR)

(21) Appl. No.: 15/927,110

(22) Filed: Mar. 21, 2018

Related U.S. Application Data
(60) Provisional application No. 62/474,972, filed on Mar. 22, 2017.

(51) Int. Cl. A61N 1/04; A61N 1/36

(52) U.S. Cl. CPC A61N 1/0456; A61N 1/36021; A61N 1/3603; A61N 1/0484; A61N 1/0492

(57) ABSTRACT

A pulsed transdermal electrical stimulation (pTES) apparatus targeting cervical nerves on the back of the neck includes a pulsed electrical stimulation neckband and an electrode patch, the pulsed electrical stimulation neckband including a neckband body configured to be worn on the neck; a stimulation generator configured to generate signals for pulsed electrical stimulation; a patch connector provided on the neckband body in connection with the stimulation generator; and a controller configured to control the stimulation generator, and the electrode patch including a patch body for being attached on the location of the back of the neck; an electrode connector provided on the patch body and detachably combined with the patch connector; and electrodes for providing the signals for pulsed electrical stimulation of the stimulation generator transferred from the electrode connector to a skin of the back of the neck on which the patch body is attached.

### FIELD

The present application relates to apparatuses (e.g., systems and devices) and methods for noninvasive neuromodulation by targeting cervical nerves using transdermal pulsed electrical stimulation. The neuromodulation apparatuses may be used to improve sleep, treat insomnia, and mitigate anxiety.

### BACKGROUND

Targeting peripheral nerves innervating muscles of the body (arms, back, and legs, for example) or cranial nerves innervating the face or head (facial and/or trigeminal nerves, for example) to treat pain (including headache) or to provide face-improvement are known and generally referred to as transcutaneous electrical nerve stimulation (TENS) devices. However, comfortable transdermal electrical stimulation systems and methods that target the cervical plexus including spinal and cranial nerves located near the dorsal surface of the neck have not been previously described and/or are less than ideal for efficacy of neuromodulation and ease of self-comfortable use. Targeting these peripheral nerves has great potential for consumer and therapeutic applications. The efficacy of apparatuses for cervical nerve stimulation relies on a foundation of functional neuroanatomy of cervical nerve targets and their projections to brainstem networks involved in physiological arousal and stress. A more detailed discussion of the nerves targeted and proposed mechanisms of action are described below as background to support the features of the devices and character of the methods of use described herein.

FIG. 1 is a schematic showing afferent and efferent nerve circuitry of the cervical plexus. The schematic illustrates a diagram of the major peripheral nerves comprising the cervical plexus. Besides cervical spinal nerves cervical (C) vertebrae 1-4 (C1-C4) there are several cranial nerves (CN's) associated with the cervical plexus including CN IX (accessory nerve), CN X (vagus nerve), and CN XII (hypoglossal nerve). The cervical plexus also shares robust convergent inputs with CN V and the spinal nucleus of the trigeminal sensory complex. The efferent pathways regulate descending motor activity while the afferent pathways provide strong ascending sensory inputs to key arousal centers of the brain located in the pons and midbrain of the brainstem. The inventions described herein target the cervical plexus and associated circuitry as indicated above. The cervical plexus is formed by a collection of nerves located at the base of the skull running down the neck from vertebrae C1-4. Each of the cervical nerves forming the cervical or craniocervical plexus communicates with one another in a superior-inferior fashion close to their origins, thus C1 receives communicating fibers from C2, C3 from C2, and C4 from C3. These communicating fibers are contributions from the sympathetic trunk (sympathetic nervous system) to the cervical plexus. These fibers are gray rami (meaning blood vessel accompanied) descending from the superior cervical ganglion (the largest of the three cervical ganglia). With the exception of C1, each cervical branch divides into an ascending branch and a descending branch, and unite with branches of the adjacent cervical nerve to form loops, for example, the loop formed between C2 and C3 that contributes branches to the ansa cervicalis. Those loops and the branches from them comprise the cervical plexus.

Branches of the cervical plexus include mixed motor fibers innervating muscles and sensory cutaneous nerves innervating the skin of the anterolateral neck, the superior part of the thorax (superolateral thoracic wall) and scalp between the auricle (pinna; outer ear) and the external occipital protuberance located at the base of the skull.

Below we provide a thorough description of the afferent sensory circuits making up the cervical plexus. The pulsed Transdermal Electrical Stimulation apparatus (hereinafter this is called ‘pTES apparatus’) of the present invention delivers pTES designed to target and modulate afferent sensory and proprioceptive pathways, we also provide a brief discussion of motor circuits making up the cervical plexus for completeness since some of a cervical pTES apparatus' effects may involve activity modulation of these circuits. We then discuss the ascending circuitry, which transmits information from the cervical plexus and periphery to the brain and central nervous system. The description of this neurophysiological mechanism circuitry rising primarily through the spinal cord, medulla, pons, and midbrain to cerebrum region shows how a cervical pTES apparatus operates to stimulate or trigger endogenous neurophysiological and psychophysiological relaxation in users via brainstem targets of cervical nerve pathways.

The groups of nerves or nerve bundles targeted by a cervical pTES apparatus include cervical plexus and vagus nerve, and the cervical plexus includes greater auricular nerve, hypoglossal nerve, transverse cervical nerve, and phrenic nerve.

The sensory (posterior or cutaneous) branches of the cervical plexus emerge around the middle of the posterior border of the sternocleidomastoid muscle (roughly the midpoint on the side of the neck located towards the back of the head inline with the back of the ear). This area is clinically significant and recognized as the nerve point of the neck, where anesthetics can be injected to achieve cervical nerve blocks to alleviate head pain (including headache), neck pain, face pain, tooth pain, and shoulder pain for example. Positions indicated by numbers 11-14 in FIG. 4(a) approximately show a region of delivery of stimulation of a cervical pTES apparatus as it is positioned on the back of the neck in between the locations of these nerve points as shown in FIG. 1.

There are four main pairs of sensory branches of the cervical plexus originating from the two loops formed between the ventral rami of C2 and C3, and C1 and C4. The branches of the loop between C2 and C3 are: the Lesser Occipital nerve (formed by C2); the Great Auricular nerve (formed by C2 and C3); and the Transverse Cervical nerve (formed by C2 and C3). The branches of the loop between C3 and C4 are the Supraclavicular nerves (formed by C3 and C4).

The Lesser occipital nerve is formed by the second cervical nerve (C2) only, and a branch of fasciculus widely distributed in the skin of the neck and the scalp posterosuperior to the clavicle. The Great Auricular Nerve is the sensory branch, which originates from the C2 and C3 nerves. It courses upwards in a diagonal fashion and crosses the sternocleidomastoid muscle onto the parotid gland, where it divides and innervates the skin over the parotid gland, the posterior aspect of the auricle, and an area of skin extending from the angle of the mandible of the mastoid process. The Transverse cervical nerve is formed by axons from the C2 and C3 nerves and distributed in the skin covering the anterior triangle of the neck. The Supraclavicular nerve is formed by C3 and C4 nerves and emerges as a common trunk under cover of the sternocleidomastoid muscle. It sends small branches to the skin of the neck. Some of those branches (supraclavicular) are also distributed cross the clavicle to the skin over the shoulder. Besides these main sensory branches of the cervical plexus, as illustrated in FIGS. 1 and 2 there are several sensory components of cranial nerves interconnected within the plexus including CN V (trigeminal nerve), CN IX (accessory nerve), CN X (vagus nerve), and CN XII (hypoglossal nerve). The phrenic nerve also transmits sensory information from the diaphragm through the cervical plexus to the brain as discussed below.

The motor branches of the cervical plexus form the ansa cervicalis, which is a nerve loop innervating the infrahyoid muscles in the anterior cervical triangle, and also form the phrenic nerve which supply the diaphragm and the pericardium of the heart (FIG. 1). The motor nerves forming the ansa cervicalis are Geniohyoid nerves (C1), Thyrohyoid nerves (C1), Omohyoid nerves (C1-C3), Sternohyoid nerves (C1-C3), and Sternothyroid nerves (C1-C3; FIG. 1).

The phrenic nerve originates chiefly from C4, but also receives contributions from C3 and C5. It is formed at the superior part of the lateral border of the anterior scalene muscle, at the level of the superior border of the thyroid cartilage. The phrenic nerve contains motor, sensory, and sympathetic nerve fibers. It provides the sole motor supply to the diaphragm and receives sensory information from its central region. In the thorax, the phrenic nerve innervates the mediastinal pleura and pericardium of the heart. The phrenic nerve descends obliquely across the anterior scalenus muscle, deep to the prevertebral layer of deep cervical fascia and the transverse cervical and suprascapular arteries. It runs posterior to the subclavian vein and anterior to the internal thoracic artery as it enters the thorax.

FIG. 2 shows afferent neural pathways of the cervical plexus ascending through the trigeminal sensory nuclear complex to the brainstem and higher brain regions. A, The illustration shows the trigeminal sensory nuclear complex (TSNC) as shaded regions in the spinal cord and brain stem. The TSNC is the principal or primary nucleus receiving inputs from the trigeminal nerve (CN V) and its branches (V1-V3). The spinal nucleus of the TSNC receives inputs from C1-C3 spinal nerves, as well as other cranial nerves such as the facial nerve (CN VII), accessory nerve (CN IX), vagus nerve (CN X) and hypoglossal nerve (CN XII). From the TSNC, the afferent information carried by these peripheral nerves is transmitted ultimately through the ventral posteromedial (VPM) region of the thalamus and sensory cortex. FIG. 2(b) shows where the cervical plexus and some trigeminal nerves enter the spinal nucleus of the TSNC in relation to the spinal cord, pons and brainstem. There is a significant degree of cross talk between the inputs of the TSNC and other key nuclei receiving similar primary cranial and cervical nerve information. These regions illustrated include the nucleus ambiguous and nucleus of the solitary tract. The TSNC provides direct outputs to the thalamus, as well as to the locus coeruleus, raphe nuclei, and other major arousal centers of the brain.

As shown in FIG. 2, the trigeminal sensory nuclear complex (TSNC) receives monosynaptic inputs from the cervical plexus and trigeminal nerves. In turn the TSNC transmits information to collateral and ascending pathways to multiple brain regions that regulate arousal and coordinate neurobehavioral engagement with the environment, such as the thalamus, the superior colliculus, the cerebellum, and several regions of the ascending reticular activating system (RAS) including the locus coeruleus (LC) and pedunculopontine tegmental nuclei (PPT).

It is already known how sensory inputs gate arousal and regulate sleep-wake cycles by influencing the activity of the ascending reticular activating system and its neuromodulatory effectors. Two of the most important complimentary and opposing arousal neuromodulatory systems of the brain are i) causing arousal (wakefulness/alertness) and coordinating attention in response to incoming sensory stimuli and ii) triggering sleep onset. One of the chief nuclei of the ascending reticular activating system (RAS) is the locus coeruleus (LC), which receives inputs from many integrative sensory nuclei in the brainstem including the trigeminal sensory nuclear complex (TSNC). FIG. 3(a) is a composite schematic showing the role of LC activity—which produces norepinephrine (noradrenaline (NA)) in response to incoming sensory stimuli including from the TSNC—in physiological arousal. The FIG. 3(a) shows how attention and general task performance relate to LC activity and NA concentration. Low LC activity and NA concentrations correspond to low levels of arousal or relaxed, disengaged states. On the other hand, high LC activity and NA concentrations correspond to a high arousal or stressed states that produce high levels of distractibility and poor performance. Components of the RAS including the LC, raphe nuclei (R), and pedunculopontine tegmental (PPT) nuclei that produce NA, serotonin (5-HT), and acetylcholine (ACh) respectively, and play crucial roles in maintaining wakefulness (arousal/alertness/attention) by suppressing the activity of other brain regions that in turn serve as sleep trigger centers by inhibiting RAS activity under appropriate conditions. This is known as mutual inhibition and provides the basis of the “flip-flop” model of sleep-wake transitions shown in FIG. 3(b). FIG. 3(b) shows how mutual inhibition affects the suppression of LC activity and inhibition of NA signaling, critical events required for sleep onset.

The RAS is a collection of nuclei and circuits that sort, filter, integrate, and transmit incoming sensory information from the brainstem to the cortex to regulate sleep/wake cycles, arousal/alertness, attention, and sensorimotor behaviors. The endogenous neuromodulatory actions of the RAS on consciousness and attention are orchestrated by at least three distinct sets of brainstem nuclei that include cholinergic neurons of the PPT, noradrenergic (NA) neurons of the LC, and serotonergic (5-HT) neurons of the raphe nuclei (RN).

Through a network of connected brainstem nuclei in the pons and midbrain (FIGS. 5D and 6B), sensory inputs first act upon the brain to engage ascending RAS networks, which generate global arousal (“waking”), alerting, and orienting cues as parsed sensory information projects through thalamic pathways onto the cortex for additional processing and integration. More specifically RAS networks (including neurons of the LC, PPT, RN) act to gate information flow from the sensory environment to the cortex. The RAS rapidly triggers neurobehavioral transitions across different states of awareness and consciousness in an activity-dependent manner. For example, neurons of the PPT can differentially mediate REM sleep states depending on their spiking rates and neurons of the LC can trigger sleep/wake transitions.

Mechanisms of several neuropsychiatric conditions and disorders, such as insomnia, anxiety, depression, post-traumatic stress disorder (PTSD), and attention deficit hyperactivity disorder (ADHD) are related to abnormal activity of ascending RAS networks. There are numerous lines of evidence demonstrating insomnia is a “waking” disorder (hyper-arousal) of RAS networks rather than a sleep disorder per se. Similar hyper-arousal hypothesis have also recently received support whereby PTSD, anxiety, some attention disorders are different manifestations of hyper-adrenergic activity and/or pathologically high levels of sympathetic activity (for example, chronic stress).

Sleep-wake cycles are tightly regulated by RAS activity and opposing inhibitory network interactions. The ascending reticular activating system (RAS) including the locus coeruleus (LC), pedunculopontine tegmental nuceli (PPT), and raphe nuclei (RN) serve to establish and maintain conscious awareness, as well as attention to stimuli and arousal during wakefulness by transmitting noradrenaline (NA; norepinephrine), acetylcholine (ACh), and serotonin (5-HT) to vast regions of the brain from the LC, PPT, and RN respectively. The tuberomammillary nucleus (TMN) also utilizes histamine in a similar manner to regulate arousal during wakefulness. Another neurohormonal signal that stimulates these arousal regions is orexin (ORX). When the arousal brain centers like the LC are active during wakefulness, they inhibit the activity of sleep triggering neurons located in the ventrolateral preoptic area (VLPO) as shown on FIG. 3(b). As shown on FIG. 3(b), when activity in neurons of the LC and other arousal regions decrease, the activity of VLPO neurons increase and begin to actively suppress the activity of arousal regions. This process is known as mutual inhibition and serves as the basis for the all or none nature of the flip-flop model of sleep-wake cycles.

The “flip-flop” model of sleep onset describes how RAS nuclei (LC, RN, and PPT) engage in mutual inhibition with other key brain nuclei to rapidly regulate conscious awareness across sleep-wake transitions (FIG. 7). As previously described, one of the primary functions of the LC is to keep the brain and body constantly prepared for or engaged in action by allocating and coordinating attention and arousal in response to all incoming sensory information first processed in the brainstem. The LC does so by transmitting the monoamine NA (norepinephrine; NE) to about 85% of the brain where it acts on different types of receptors including alpha (α) and beta (β) adrenergic receptor subtypes. LC activity and NE transmission act as real-time central regulators of baseline brain activity. They exert many dynamic filtering operations on brain activity that are required to optimize signal-to-noise relationships amongst neural networks for the effective conscious processing of incoming sensory information and awareness.

The essential alerting and orienting functions of the LC also influence sympathetic nervous system (SNS) activity and underlie the neurophysiological foundations of the commonly known “fight-or-flight” response. Under normal sensory processing these actions allow the brain and body to be engaged to perform general tasks as shown in FIG. 2. However, if a threatening sensory stimulus is presented (for example, the sound of an alarm or sight of a vicious animal) then the activity of the LC surges to instantly prepare the body to escape or defend itself. Returning the body to calm or relaxed states after a fight-or-flight response, as well as from persistent ongoing processing of sensory stimuli (daily tasks; stress) typically takes a considerably longer time than it takes to become aroused, attentive, or alert. This is why people generally require several tens of minutes to calm down after working or studying to relax enough and fall asleep. The deepest stages of sleep typically occur twenty or more minutes after sleep onset. Somewhat paradoxically and due to the intrinsic nature of flip-flop style mechanisms of sleep onset however, the transition from awake to sleep does occur within seconds. The major point is that suppression of LC and SNS activity is an essential requirement and critical trigger for sleep onset to occur. Stimulation, suppression, and perturbation of LC activity via cervical plexus modulation of TSNC networks with a pTES device as described herein may result in a shifting of brain networks for dynamical sleep-wake responsible for triggering sleep onset to help or cause a subject to fall asleep.

As shown in FIG. 3, the LC suppresses sleep circuit activity during awake behaviors in response to sensory inputs. Conversely, LC activity is suppressed by the brain's sleep circuitry to trigger sleep onset. Other arousal networks are also involved in regulating sleep-wake behaviors. For example, the pedunculopontine tegmental (PPT) nucleus uses the neuromodulator ACh to play a crucial role in gating information flow between the thalamus and the cerebral cortex. PPT neurons are highly active during wakefulness and during REM sleep and experience low activation during NREM sleep. The PPT neurons are regulated by mutual inhibition with sleep circuits as described below and illustrated in FIG. 3(b). The dorsal and median raphe nuclei (RN), ventral periaqueductal grey matter, and tuberomammillary nucleus (TMN) also each produces different neuromodulators that regulate arousal by acting on cortical and subcortical networks. Neurons in the RN, ventral periaqueductal grey matter (vPAG), and TMN produce serotonin (5-HT), dopamine (DA) and histamine respectively. Interestingly, many anti-histamine drugs (Benadryl, for example) block the histaminergic arousing signal and cause sleepiness. Another collection of neurons in the lateral hypothalamus produce a neurotransmitter called orexin (ORX; also known as hypocretin), which directly stimulates LC, PPT, and RN arousal centers, as well as the cerebral cortex. It is known that ORX serves as a key signaling molecule that integrates metabolic, circadian, and sleep debt influences to determine whether an individual should be asleep or awake and active. The majority of so-called “sleep neurons” however are located in the ventrolateral preoptic area (VLPO). These sleep neurons keep an inactivation until an individual shows a transition from waking to sleep when the VLPO neurons become active (due in part to decreased LC activity) and suppress LC, TMN, and RN activity. The sleep neurons in the preoptic area receive inhibitory inputs from some of the same regions they inhibit, including the LC, TMN, and RN. Thus, they are inhibited by NE, histamine, and 5-HT. This mutual inhibition is thought to provide a basis for establishing and triggering periods of sleep and waking.

Transitions between the bi-stable states of wakefulness and sleep occur relatively quickly, often in just seconds. The neurological mechanisms that control these rapid transitions are thought to be analogous to a “flip-flop” electrical circuit. A flip-flop in an electrical circuit can assume one of two states, usually referred to as “on” or “off”. Similarly, sleep neurons are either active and inhibit the wakefulness neurons, or the wakefulness neurons are active and inhibit the sleep neurons. Because these regions are mutually inhibitory, it is impossible for neurons in both sets of regions to be active at the same time. This flip-flop, switching from one state to another quickly, can be unstable and sensitive to perturbation. The same flip-flop analogy is also used to sometimes describe brain mechanisms involved in switching between REM sleep and NREM stages of sleep. Different neurotransmitters and different groups of neurons, such as ACh and NE from the PPT and LC respectively are involved in the transitions between REM and NREM sleep.

### SUMMARY OF THE DISCLOSURE

In general, described herein are wearable cervical pulsed transdermal electrical stimulation (pTES) apparatuses for modulating cervical nerves and reducing sympathetic nervous system activity. The apparatuses as described herein may be used by a subject autonomously and may induce states of calmness, sleep, reduced muscular activation, reduced heart rate, increased facial temperature, etc. The devices as described herein generally comprise a neurostimulator module, a skin electrode patch, and a form factor that enables the pulsed electrical stimulation neckband to be portable and/or wearable. The cervical pTES apparatuses described herein have been designed to suppress and modulate the endogenous activity of RAS circuits including the LC by modulating the cervical plexus and cranial nerves in the dorsal region of the neck transdermally. These cervical and cranial nerves transmit primary sensory information directly to the TSNC and influence the activity of the LC and ascending RAS. By suppressing activity in these ascending arousal networks, pTES as described herein induces physiological relaxation via suppressing sympathetic nervous system (SNS) activity. These effects can be observed acutely as a decrease in heart rate (HR), shifts in heart rate variability (HRV), and increases in skin temperature (particularly facial temperature). The effects may also be accompanied by a slowing in respiration (perhaps due to effects on the phrenic nerve) and a general sense of psychological relaxation. If pTES suppresses RAS or LC activity sufficiently, then dominant activity mechanisms of the flip-flop circuits are occurred. This should cause the LC to become more inhibited by the VLPO than the VLPO to become inhibited by the LC, thereby conditions for sleep onset can be optimized. Further, a general reduction in activation of sympathetic nerve prior to sleep onset should lead to an improvement in sleep quality by reducing the number of wakes in sleep and inducing deep sleep for restorative.

Traditional TENS devices operate at a stimulus frequency of about 80 to 130 cycles per second (Hertz; Hz), which is an optimal frequency band for stimulating neuromuscular activity. The invention described herein operates at frequencies from 350-500 Hz, which avoids (or mildly suppresses) neuromuscular activation while enabling modulation of afferent sensory and proprioceptive fibers. Likewise, the use of 350-500 Hz pulsed transdermal electrical stimulation (pTES) waveforms minimizes or eliminates the activation of pain fibers and pathways, which typically respond up to about 200 Hz. Therefore, the choice of pTES frequencies provides a safer and more comfortable experience for users by minimizing muscle stimulation and pain fiber activation compared to traditional TENS and other substantially equivalent approaches like electrical muscle stimulation (EMS), neuromuscular electrical stimulation (LAMES), and powered muscle stimulation (PMS).

According to an embodiment of the present invention, a pulsed transdermal electrical stimulation (pTES) apparatus targeting cervical nerves on the back of the neck comprises a pulsed electrical stimulation neckband and an electrode patch.

The pulsed electrical stimulation neckband includes a neckband body configured to be worn on the neck; a stimulation generator configured to generate signals for pulsed electrical stimulation, wherein the stimulation generator is provided on a location of the back of the neck in the neckband body; a patch connector provided on the neckband body in connection with the stimulation generator; and a controller configured to control the stimulation generator.

The electrode patch includes a patch body for being attached on the location of the back of the neck; an electrode connector provided on the patch body and detachably combined with the patch connector so as to electrically connect to the patch connector; and electrodes for providing the signals for pulsed electrical stimulation of the stimulation generator transferred from the electrode connector to a skin of the back of the neck on which the patch body is attached.

Wherein the patch body forms electrode arranging portions corresponding to four regions to be stimulated targeting cervical nerves on the back of the neck, the electrodes include a first electrode, a second electrode, a third electrode and a forth electrode provided on each of the electrode arranging portions, and the first electrode and the second electrode make a pair of electrodes, and the third electrode and the forth electrode make another pair of electrodes, wherein each the pair of electrodes provide a pulsed electrical stimulation by a bipolar manner.

Also, wherein the electrode connector includes a first connector connected with the first electrode and the third electrode for assigning a polarity to the first electrode and the third electrode, and a second connector connected with the second electrode and the forth electrode for assigning the opposite polarity to the second electrode and the forth electrode, the signals for pulsed electrical stimulation of the stimulation generator are transferred to each of the electrodes through the first connector and the second connector, and the pulsed electrical stimulation energies between the first electrode and the second electrode and between the third electrode and the forth electrode are respectively transferred to the four regions to be stimulated targeting cervical nerves on the back of the neck.

Also, wherein each of the electrodes provides at least one of polar members for forming an electrode providing an electrical stimulation, and the patch body includes a conductive pattern layer coated by conductive material for electrically connecting with the electrode connector and at least one of the polar members in each of the electrodes.

Also, wherein the each of the electrodes includes a conductive hydrogel layer for sticking to the skin and delivering the electrical stimulation to the regions on the back of the neck through at least one of the polar members.

According to another embodiment of the present invention, a pulsed transdermal electrical stimulation (pTES) apparatus targeting cervical nerves on the back of the neck comprises, a pulsed electrical stimulation neckband configured to deliver pulsed symmetric charge balanced electrical stimulation having a frequency between 350 and 500 Hz, a pulse width of 200 to 250 microseconds, an pulse interval of 125 to 375 microseconds, and a peak amplitude of 0.5 to 10 mA; an electrode patch configured to connect electromechanically to the pulsed electrical stimulation neckband and provide two or more electrically conductive electrodes to a user's skin; and a user control for activating the pulsed electrical stimulation neckband to deliver electrical stimulation transdermally to a subject.

Also, wherein the heart rate of a user decreases in response to electrical stimulation.

Also, wherein the heart rate variability of a user changes in response to electrical stimulation.

Also, wherein the skin temperature on a user's face increases in response to electrical stimulation.

Also, wherein the electrode patch has four active areas.

Also, wherein the active areas of an electrode assembly are round and roughly symmetrically positioned across the midline of the spine on the neck.

According to an embodiment of the present invention, a control method of a pTES apparatus targeting cervical nerves on the back of the neck, the method comprises, electromechanically connecting with a pulsed electrical stimulation neckband and an electrode patch as positioning symmetrically on the back of the neck the electrode patch and wearing the pulsed electrical stimulation neckband; and controlling a stimulation generator of the pulsed electrical stimulation neckband to deliver pulsed symmetric charge balanced electrical stimulation having a frequency between 350 and 500 Hz, a pulse width of 200 to 250 microseconds, an pulse interval of 125 to 375 microseconds, and a peak amplitude of 0.5 to 10 mA so as to transfer the stimulation to cervical nerves on the back of the neck through the electrode patch.

### BRIEF DESCRIPTION OF THE DRAWINGS

FIGS. 1 to 3 are schematics for describing background regarding the present invention.

FIG. 4 illustrates (a) which shows the anatomical targeting of the cervical plexus by a cervical pTES apparatus according to an embodiment of the present invention with a posterior view, and (b) and (c) which show a stimulated cervical nerves and an upper region of cranial nerve with a posterior view.

FIG. 5 shows an electrode patch used to the pTES apparatus according to an embodiment of the present invention.

FIG. 6 shows a pulsed electrical stimulation neckband used with the electrode patch as shown in FIG. 5 in the pTES apparatus according to an embodiment of the present invention.

FIG. 7 illustrates (a) which shows a connection with the pulsed electrical stimulation neckband and the electrode patch in the pTES apparatus according to an embodiment of the present invention, and (b) which shows a state of the electrode patch connected with the pulsed electrical stimulation neckband.

FIG. 8 illustrates (a) and (b) which show detailed configurations of the electrode patch as shown in FIG. 5.

FIG. 9 illustrates (a) which shows a state of the electrode patch as shown in FIG. 8 attached to regions to be stimulated on the back of the neck, and (b) which shows a state of combination of the attached electrode patch and the pulsed electrical stimulation neckband as shown in FIG. 6.

FIGS. 10 and 11 show a result of comparing with heart rate data from subjects treated with cervical pTES (top) and sham stimulation (bottom), normalized and averaged heart rate data, and heart rate data as Z scores.

FIGS. 12 to 14 show average heart rate data before, during, and after either sham (left) or cervical pTES treatment (right), and various heart rate variability metrics before, during, and after either sham (left) or pTES treatment (right).

FIG. 15 illustrates (a) which shows forward-looking infrared (FLIR) imaging data before, during, and after cervical pTES treatment, and (b) which shows average temperature at various facial locations for subjects treated with cervical pTES.

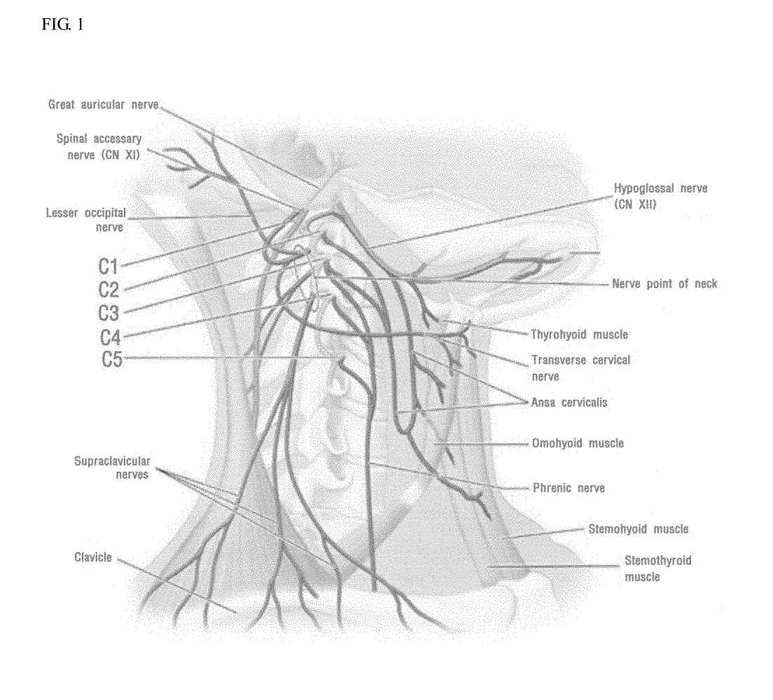

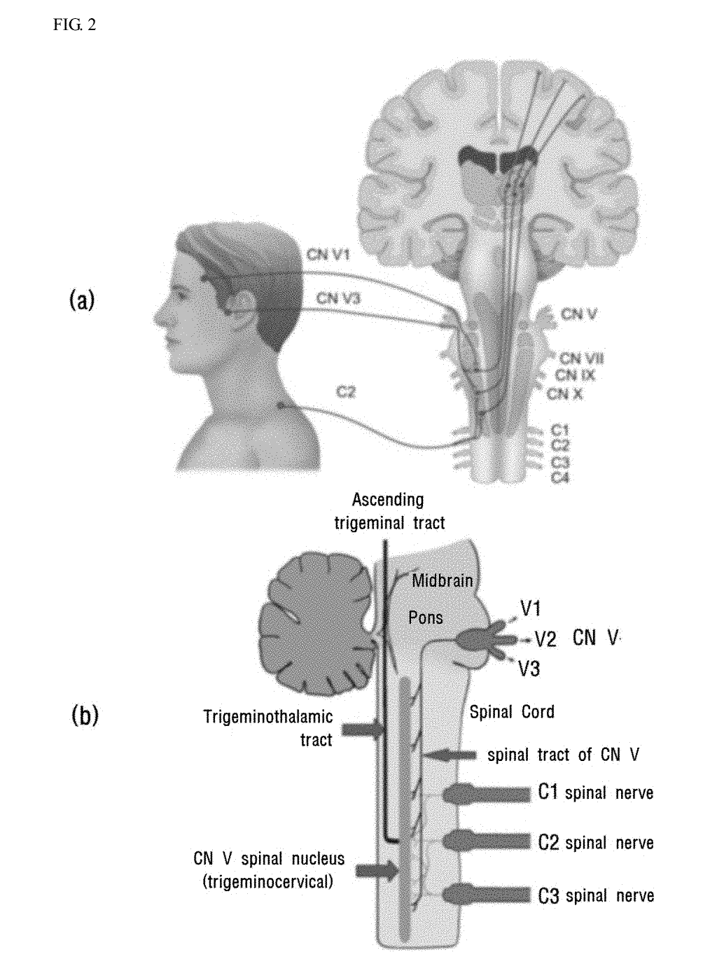

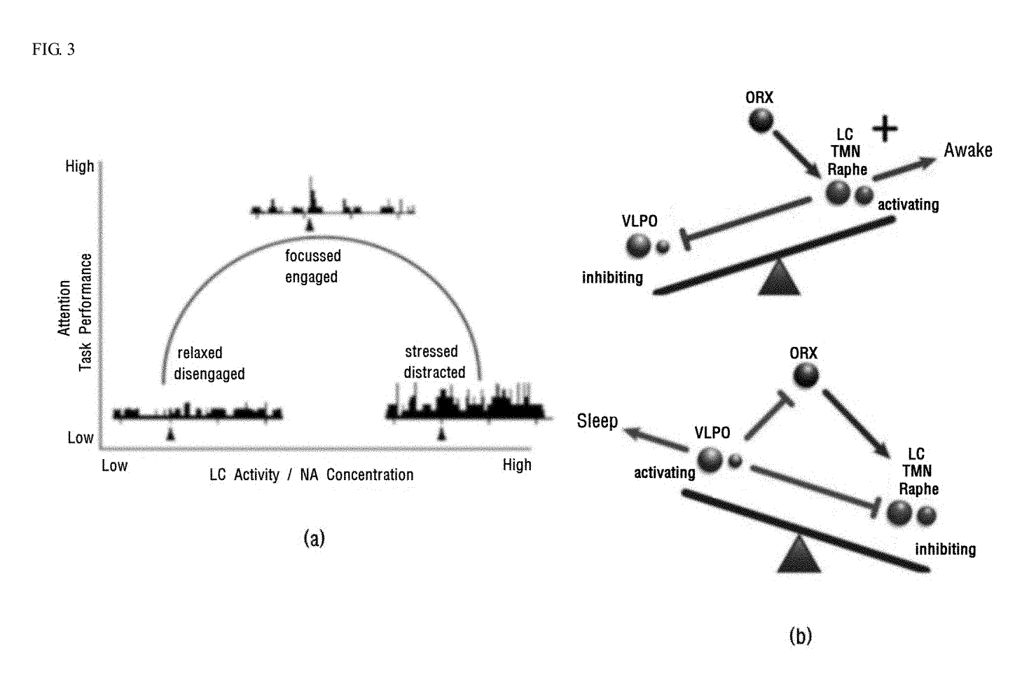

### DETAILED DESCRIPTION

Traditional TENS devices operate at a stimulus frequency of about 80 to 130 cycles per second (Hertz; Hz), which is an optimal frequency band for stimulating neuromuscular activity. The invention described herein operates at frequencies from 350-500 Hz with biphasic, charge balanced pulsed waveforms (pulse widths 200-250 microseconds; interpulse intervals 125-375 microseconds). Pulse amplitudes are in the mA range (generally 0.5 to 10 mA pulse amplitude, though higher intensities up to 30 mA may be used in some embodiments.) These device characteristics avoid (or mildly suppress) neuromuscular activation while enabling modulation of afferent sensory and proprioceptive fibers. Likewise, the use of 350-500 Hz pulsed transdermal electrical stimulation (pTES) waveforms with these parameters minimizes or eliminates the activation of pain fibers and pathways, which typically respond up to about 200 Hz. Moreover, the charge balanced nature of the waveform minimizes pH changes known to occur in the skin with direct current stimulation. Therefore, the choice of pTES parameters provides a safer and more comfortable experience for users by minimizing muscle stimulation and pain fiber activation compared to traditional TENS and other substantially equivalent approaches like electrical muscle stimulation (EMS), neuromuscular electrical stimulation (MMES), and powered muscle stimulation (PMS). Yet the device parameters described herein are effective for delivering neuromodulation to the cervical plexus and, as described below, significantly suppress sympathetic nervous system activity, presumably through brainstem circuits as described in the background section above.

The cervical pTES apparatuses described herein deliver safe extra-low voltage, pulsed electrical currents to the skin of the back of the neck that may also be described to embody a medical device depending on the intended use or application. The cervical pTES apparatuses modulate the activity of the peripheral nerves of the cervical plexus and neck using weak electrical pulses transmitted through the skin. The cervical pTES apparatuses described herein are intended to deliver pulsed transdermal electrical stimulation (pTES) that promote relaxation for the body and mind to improve rest and sleep.

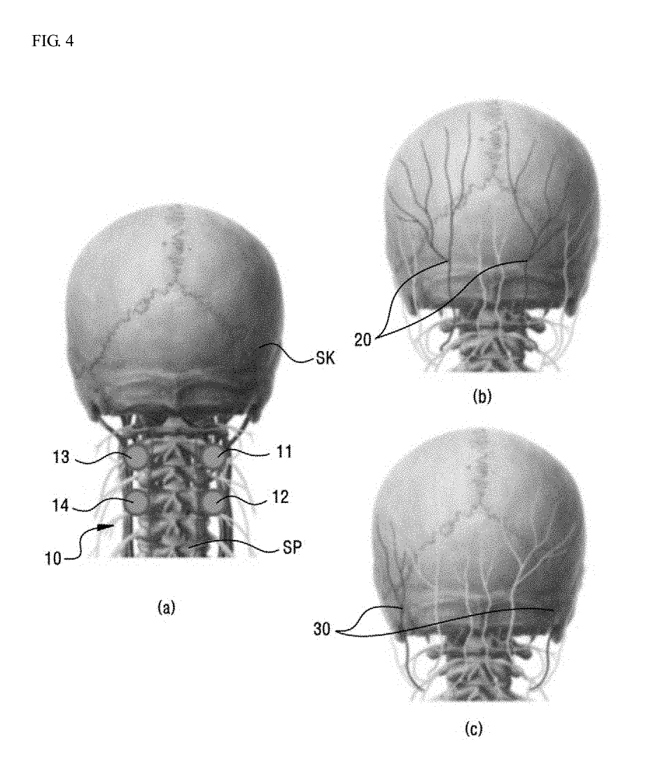

FIG. 4 shows anatomical targeting of the cervical plexus by the cervical pTES apparatuses as described herein. As illustrated in FIG. 4(a), a pTES cervical stimulation apparatus targets four regions on the back of the neck (the four regions are regions to be stimulated (11-14)) and the underlying cervical plexus 10 circuitry as indicated by the numbers 11 to 14, and the greater occipital nerve 20 and the lesser occipital nerve 30 as cranial nerves to be influenced by stimulation of the regions to be stimulated. The regions to be stimulated (11-14) of the cervical plexus as shown in FIG. 4(a) preferably shows the C2-C4 region of the neck. The nerve targeted by the stimulation of the regions to be stimulated (11-14) is the cervical plexus which includes hypoglossal nerve, great auricular nerve, spinal nerve C2, phrenic nerve, transverse cervical nerve, accessory nerve to the trapezieus muscle, and spinal nerves C1, C3, and C4.

### Example: Cervical pTES Apparatus

A cervical pTES apparatus according to an embodiment of the present invention comprises a pulsed electrical stimulation neckband, an electrode patch, and, optionally, proprietary software and cloud communication protocols enabling a user to control or regulate other connected devices within their environment. Other optional apparatus features provide device WiFi communication and synchronization of user data with data from other wearable devices, sleep trackers, or fitness trackers.

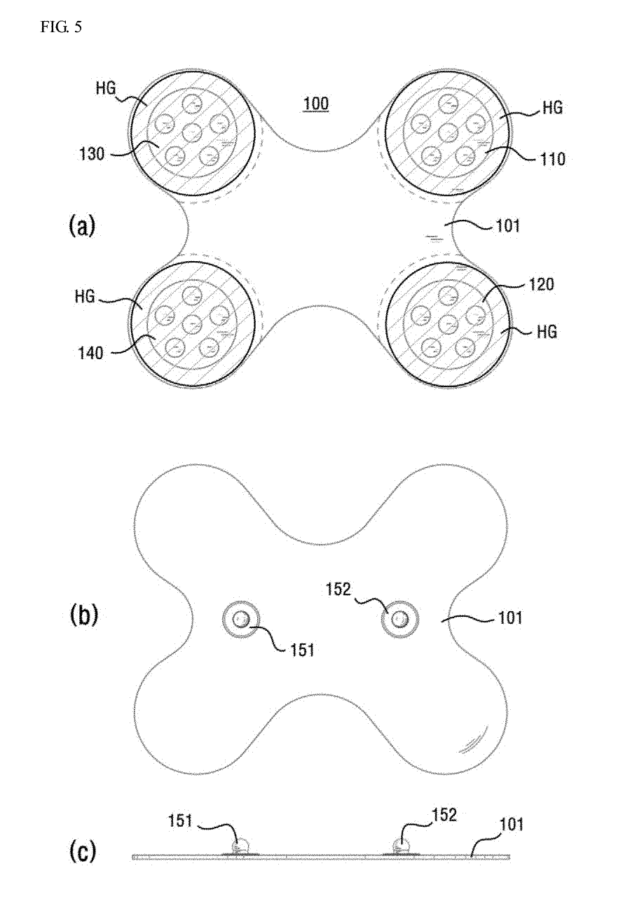

FIGS. 5 to 8 show an embodiment of the present invention, a pulsed Transdermal Electrical Stimulation (pTES) apparatus.

A pTES apparatus according to an embodiment of the present invention delivers a constant current under user control with a maximum current output of 9 mA into a 500-ohm load. Currents are delivered to the tissues (including nerves) of the back of the neck using four equally sized transdermal electrodes each having a diameter of 2.54 cm (area ˜=5 cm²). The anodic and cathodic phases of current pulses are each distributed evenly across one of two electrode pairs. The maximum current density is <1 mA/cm². Current output variation is <10%. The maximum output voltage of an exemplar cervical pTES apparatus is 35V. The peak pTES frequency of programmable operation is 1 kHz. Available pTES waveforms presently operate at a fixed frequency of either 350 or 500 Hz. Pulse widths range from 200 to 250 microseconds. Other components and features of the exemplar cervical pTES device include:

Battery: 4.2V lithium polymer

Maximum Output Voltage @ 500 Ω: 35V

Maximum Output Current @ 500 Ω: 9 mA

Stimulus Current Shape: biphasic, square, symmetrical, charge-balanced

Maximum Current Density @ 500 Ω: <1 mA/cm2

Minimum Output Frequency: 350 cycles per second (350 Hz)

Maximum Output Frequency: 500 cycles per second (500 Hz)

Minimum Pulse Width: 200 μs

Maximum Pulse Width: 250 μs

Maximum Charge per Pulse: 9 μC

Electrode Area: 4×5 cm2 (two pairs of 5 cm2 round electrodes)

Operating Temperature: 0-40 C

Number of Programs: 6

The following six waveforms are available in a pTES apparatus according to an embodiment of the present invention. Note that Waveform 5 is a constant current test and debug waveform (i.e. direct current stimulation) that is not intended for transdermal use. The selected waveform is generally retained in memory if the unit is in non-stimulation mode (LED off), so long as the battery voltage does not drop too low.

| | | Interpulse | Cycle period | |
|---|---|---|---|---|
| Waveform | Pulse Width | period | (μs)/Frequency | |
| ID | (μs) | (μs) | (Hz) | LED color |
| 0 | 250 | 125 | 2857 (350 Hz) | White |
| 1 | 250 | 250 | 2857 (350 Hz) | Green |
| 2 | 250 | 375 | 2857 (350 Hz) | Blue |
| 3 | 200 | 125 | 2000 (500 Hz) | Yellow |
| 4 | 200 | 250 | 2000 (500 Hz) | Pink |
| 5 | Constant output | No pulses | For current tests | Blinking |
| | | | | crimson |

FIG. 5 shows an electrode patch used to the pTES apparatus according to an embodiment of the present invention. FIG. 5(a) shows a surface of the electrode patch to be attached to the back of a user's neck, FIG. 5(b) shows an external surface of the electrode patch to be connected to the pulsed electrical stimulation neckband, and FIG. 5(c) shows a side of the electrode patch.

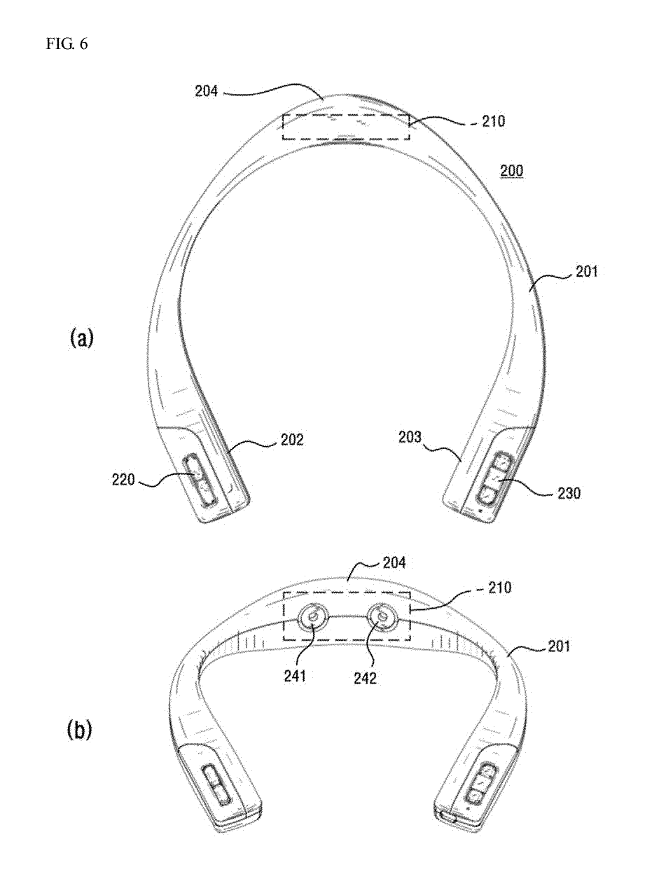

FIG. 6 shows the pulsed electrical stimulation neckband of the pTES apparatus according to an embodiment of the present invention. FIGS. 6(a) and 6(b) respectively show a plane view and a perspective view of the neckband.

The electrode patch 100 includes a patch body 101 for being attached on a targeted location of the back of the neck, an electrode connector 151 and 152 provided on the patch body 101 and detachably combined with a patch connector 241 and 242 of the neckband 200 so as to electrically connect to the patch connector 241 and 242, and electrodes (a first electrode 110, a second electrode 120, a third electrode 130 and a forth electrode 140) for providing the signals for pulsed electrical stimulation of a stimulation generator 210 of the neckband 200 transferred from the electrode connector 151 and 152 to a skin of the back of the neck on which the patch body 101 is attached.

It is called “electrodes” as a common name of the first electrode 110, the second electrode 120, the third electrode 130 and the forth electrode 140.

A conductive hydrogel HG is provided to each of the electrodes 110 to 140 so as to be easily attached to a skin and effectively deliver an electrical stimulation.

The pulsed electrical stimulation neckband 200 includes a neckband body 201 configured to be worn on the user's neck, a stimulation generator 210 configured to generate signals for pulsed electrical stimulation, wherein the stimulation generator 210 is provided on band portion 204 which is a location of the back of the neck in the neckband body 201, a patch connector 241 and 242 provided on the neckband body 201 in connection with the stimulation generator 210 and a controller (not drawn) configured to control the stimulation generator 210.

It is preferable that manipulators 220 and 230 for manipulating the stimulation generator 210 are provided on distal arms 202 and 203 which are extended from the band portion 204 in the neckband body 201 and located on a side of the user's chest when the user wears the neckband 200 around his neck.

The user can regulate intensity of the signals generated at the stimulation generator 210 and manipulate ON/OFF of the pulsed electrical stimulation neckband 200 through the manipulators 220 and 230.

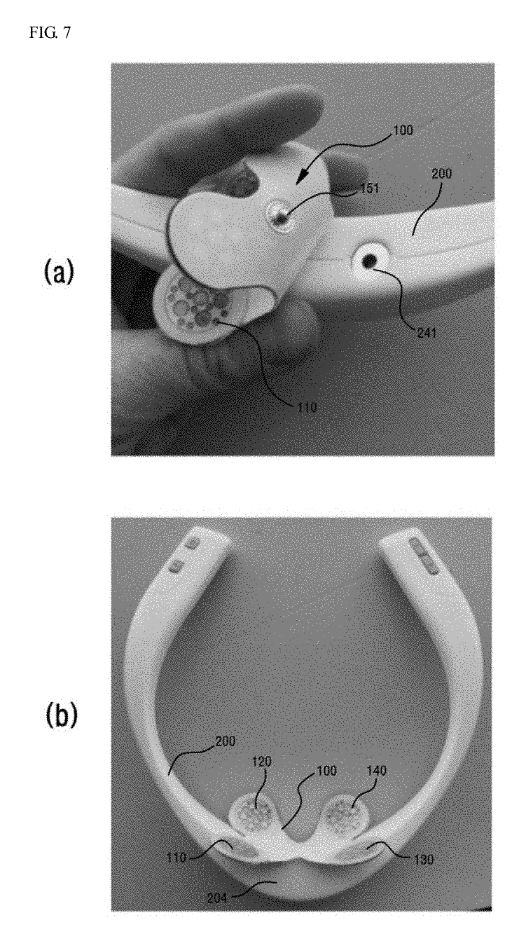

It is preferable that the patch connector 241 and 242 of the pulsed electrical stimulation neckband 200 and the electrode connector 151 and 152 of the electrode patch 100 are constituted of a magnetic combination using a magnet provided on the patch connector 241 and 242 or the electrode connector 151 and 152 so as to be easily detachable. For example, as shown in FIGS. 7(a) and 7(b), the electrode connector 151 and 152 of the electrode patch 100 can be configured for a snap and the electrode connector 151 and 152 of the electrode patch 100 can be configured for a magnetic receptacle keeping the snap in by magnetic force.

The exemplar cervical pTES apparatus is a neckband (FIGS. 21-29) made of a biomedical grade plastic and designed to be worn around a user's neck, with the two round magnet receptacles facing the back of the neck and the grey buttons facing up.

More specifically, the pulsed electrical stimulation neckband 200 of the pTES apparatus includes: four buttons (manipulators) for user control; two conductive magnetic receptacles (patch connector 241 and 242) to connect to the electrode snaps (electrode connector 151 and 152); a Li-Po ˜380 mAh battery (in the neckband, behind the snaps); a vibrating motor, LED, and WiFi antenna; and two PCBs in each of the distal arms 202 and 203 of the device, connected by a 12-channel custom conductive flex cable.

The exemplar cervical pTES apparatus according to the present invention has six modes:

(1) Stimulation: The exemplar cervical pTES apparatus LED is on, and pTES is being delivered or may be started by pressing the Power/Play button.

(2) Non-stimulation: exemplar cervical pTES apparatus is in a quiescent, ‘sleep’ mode. The device can be placed in stimulation mode (turned on) or placed in a WiFi pairing state.

(3) WiFi pairing: the WiFi chip is ready for pairing via an app.

(4) Off (low battery): exemplar cervical pTES apparatus cannot be turned on, button presses have no effect, and no previous states are stored in memory of the ST microcontroller (i.e. waveform and intensity selection).

(5) Battery charging: connected to DC power via the microUSB port. If the exemplar cervical pTES apparatus is connected to a computer, the CLI may be used in this mode.

(6) DFU: for upgrading firmware

The neckband is recharged with a standard microUSB cable plugged into a suitable charging device. The LED will slowly flash (‘breathe’) red during charging and will show solid green when the unit is fully charged

Restricting pTES to battery-operated stimulation may be a key safety feature.

The electrode patch is designed to target peripheral nerves of the cervical plexus comfortably for the waveforms configured in the Cervical pTES apparatus. The exemplar electrode patch has four round electrodes, two for each pole of the electrode on the left and right of the electrode, respectively. (The traditional terms of anodic and cathodic are not well-suited to the symmetrical, biphasic, charge-balanced waveforms delivered by a cervical pTES apparatus.)

### Internet Connectivity: WiFi Pairing Via Particle.io

The exemplar cervical pTES apparatus uses the Photon (P0) WiFi chipset from Particle (www.particle.io) and an antenna inside the neckband enclosure to connect to the Internet and send data relating to use. Pairing to a Photon chip requires the (free) Particle app, available for iOS and Android. The Photon microcontroller runs Particle firmware that can be flashed over WiFi via the Particle web console.

Communication between the cervical pTES apparatus and the cloud services platform is done with webhooks configured on the Particle website and ‘published’ via the Particle firmware flashed via WiFi onto the neckband device. There are specific webhooks that designate Cervical pTES apparatus events: ‘start’, ‘stop’, ‘up intensity’ (only publishes during a waveform), ‘down intensity’ (only publishes during a waveform), and a webhook for each of the waveform IDs. An ETL process interprets these webhook events and incorporates the cervical pTES apparatus usage data into its Picard database.

There are two methods to deliver the SSID and password to a cervical pTES apparatus:

Via the Particle app: First, open the Particle app on your mobile device. Next, with the cervical pTES apparatus in non-stimulation mode (LED off), press and hold the waveform button until the LED flashes pink (5 brief flashes every few seconds). The Cervical pTES apparatus is now in pairing mode. Choose the ‘+’ icon in the upper right of the Particle app and follow the on-screen instructions to identify the Photon WiFi network and enter the SSID and password for the desired WiFi network. Once the device has been verified, the device will connect automatically to the WiFi network when available. It is advised not to change the Photon name when providing new WiFi credentials.

Through the CLI: With the cervical pTES apparatus connected to a serial emulator (i.e. CoolTerm link), use the ‘wife’ command to provide the SSID, password, and security type. Please refer to the CLI section below for further instructions.

To check whether an exemplar cervical pTES apparatus unit is online, press and release the ‘down intensity’ button while the device is in non-stimulation mode (LED off). A one second green flash indicates that the device is connected to WiFi; a one second red flash indicates that the device is not connected to WiFi.

Cervical pTES Apparatus Connected Device Platform

The connected device functionality of the exemplar cervical pTES apparatus is provided through the Picard.io ‘Platform-as-a-service’ cloud services from Trialomics. The configuration of Picard offers three core functions:

Collection of usage for both user dashboards and company stakeholders (management business intelligence, customer support, data science, and clinical trial coordinators).

Integration with third party sensors (i.e. Fitbit for activity and sleep data)

Activation of other connected devices or other API-enabled web services that enable a user to configure (for example) their home environment (lighting, music) based on cervical pTES apparatus usage (start, stop, which waveform, intensity selected).

Picard.io is configured to permit new dashboards, functionality, and apps to be created via (1) easily-deployed in-browser Javascript and HTML; (2) a Python or R SDK; and (3) mobile apps via the Picard API.

### PCB Boards

In an exemplar cervical pTES apparatus the Power PCB board is placed on the right side of the unit (when the user is wearing the neckband) and includes the Up intensity and Down intensity buttons.

In an exemplar cervical pTES apparatus the Logic PCB board is placed on the left side of the unit (when the user is wearing the neckband) and includes the Power/Play and Waveform Select buttons.

### Custom Flex Cable

In an exemplar cervical pTES apparatus the Logic and Power boards are connected through the length of the housing by a 400 mm, 12-channel custom flex circuit cable

### Magnetic Snap Receptacles

In an exemplar cervical pTES apparatus the magnetic snap receptacles are a strong, rare-earth magnet with solder-able legs on the side enclosed within the Cervical pTES apparatus housing. Part number ROMAG 12S2 MED-NIC sold by Rome Fastener (MILFORD, Conn.).

### Electrode Patch Design Rationale and Parts

In an embodiment of the cervical pTES apparatus, the electrode patch is composed of three core layers. Beginning nearest to the skin interface is a conductive hydrogel optimized for transdermal electrical stimulation. The next layer is a conductive pattern layer, for instance a silver-coated PVC, which is covered by a nonconductive white foam that increases the durability, thickness, and rigidity of the electrode patch.

The silver layer of each electrode has a pattern of circular exclusions that increase the internal edge length of the conductive portions of the electrode. This pattern improves the uniformity of current distribution across the electrode face and improves the comfort of pTES for a given peak current intensity. Improved comfort facilitates physiological effects of relaxation mediated by inhibition of sympathetic pathways. (Consider, in contrast, noxious stimulation that would increase physiological arousal and stress.) Conductive metal snaps connect to two magnetic receptacles on the cervical pTES apparatus to deliver stimulation, and a release liner covers the hydrogel and protects it from sticking to packaging before use.

The Cervical pTES apparatus Butterfly Electrode BOM and assembly order are shown in the table below. The electrodes are delivered in individual clear zip lock bags, labeled.

| Item | | Manufac- | | | Quan- |
|---|---|---|---|---|---|
| # | Description | turer | Part number | Thickness | tity |
| 1 | Hydrogel | Axelgaard | AG-735 | 0.035″ | 1 |
| 2 | Transfer tape | PIC/CSI | #140-025 | 0.003″ | 1 |
| | | | matte litho | | |
| | | | P17 perm | | |
| 3 | Eyelet | Rome | 75 EYELET- | n/a | 2 |
| | | Fasteners | T-NIC | | |
| 4 | Silver coated | Berry | 6027 | 0.0023″ | 1 |
| | PVC | Plastics | | | |
| 5 | Transfer | Adchem | 730 | 0.003″ | 1 |
| | adhesive | | | | |
| 6 | White foam | Worthen | VN-300 | 0.028″ | 1 |
| 7 | Stud | Rome | 75 STUD- | n/a | 2 |
| | | Fasteners | Y-NIC | | |
| 8 | Release liner | 3M | 9968 | 0.0046″ | 1 |

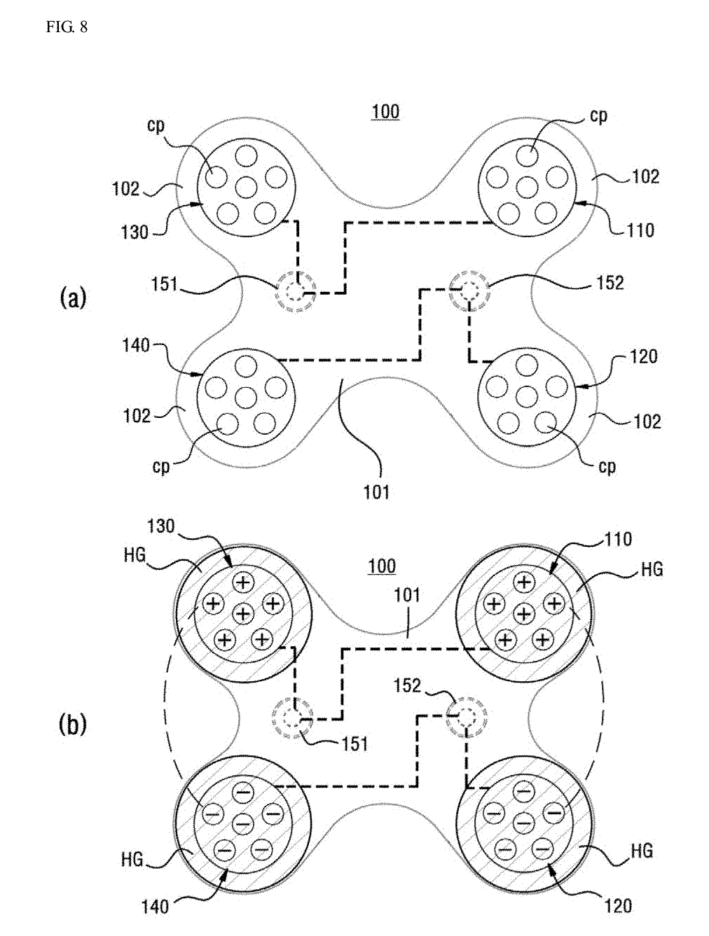

FIG. 8(a) shows each of the electrodes of the electrode patch 100 in the pTES apparatus according to an embodiment of the present invention, wherein FIG. 8(a) shows the electrode connector 151 and 152 except a conductive hydrogel HG. FIG. 8(b) shows each of the electrodes of the electrode patch 100 in FIG. 8(a) including the conductive hydrogel HG.

As shown in FIG. 8(a), the patch body 101 has electrode arranging portions 102 corresponding to four regions to be stimulated targeting cervical nerves on the back of the neck for arranging each of the electrodes 110 to 140 in diagonal locations of bilateral symmetry (the first electrode 110, the second electrode 120, the third electrode 130 and the forth electrode 140 are respectively provided on each of the electrode arranging portions 102).

At least one of polar members cp for forming an electrode providing an electrical stimulation is provided on each of the electrodes 110 to 140, and the patch body 101 includes a conductive pattern layer coated by conductive material (such as a pattern layer including silver coated PVC) for electrically connecting with the electrode connector 151 and 152 and at least one of the polar members cp.

The electrode connector includes a first connector 151 connected with at least one of the polar members cp of the first electrode 110 and at least one of the polar members cp of the third electrode 130 for assigning a polarity to the first electrode 110 and the third electrode 130, and a second connector 152 connected with at least one of the polar members cp of the second electrode 120 and at least one of the polar members cp of the forth electrode 140 for assigning the opposite polarity to the second electrode 120 and the forth electrode 140.

The signals for pulsed electrical stimulation of the stimulation generator 210 of the pulsed electrical stimulation neckband 200 are transferred to each of the electrodes through the first connector 151 and the second connector 152, and the pulsed electrical stimulation energies between the first electrode 110 and the second electrode 120 and between the third electrode 130 and the forth electrode 140 are respectively transferred to the four regions to be stimulated targeting cervical nerves on the back of the neck.

The conductive pattern layer forms a pattern by conductive material so as for the first connector 151 to connect with the polar members cp of the first electrode 110 and the third electrode 130, and for the second connector 152 to connect with the polar members cp of the second electrode 120 and the forth electrode 140.

As shown in FIG. 8(b), the first electrode 110 and the second electrode 120 make a pair of electrodes, and the third electrode 130 and the forth electrode 140 make another pair of electrodes, and each the pair of electrodes provide a pulsed electrical stimulation by a bipolar manner.

For example, as shown in FIG. 8(b), according to the first connector 151 assigns + polarity to the polar members cp of the first electrode 110 and the third electrode 130, and the second connector 152 assigns − polarity to the polar members cp of the second electrode 120 and the forth electrode 140, the first electrode 110 and the second electrode 120 make a pair of + and − electrodes, and the third electrode 130 and the forth electrode 140 make another pair of + and − electrodes so as for each the pair of electrodes to provide a pulsed electrical stimulation by a bipolar manner.

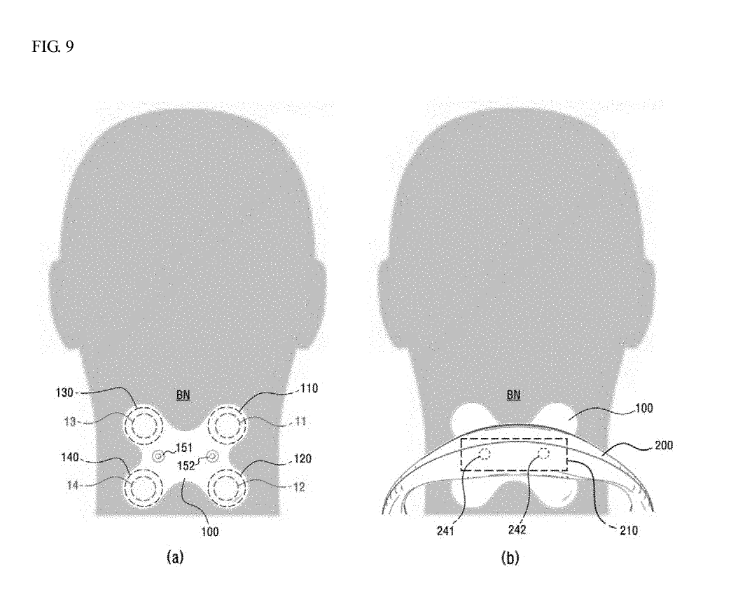

FIG. 9(a) shows a state of the electrode patch 100 attached to regions 11-14 to be stimulated on the back of the neck BN targeting cervical nerves (the first electrode 110 attached on region 11, the second electrode 120 attached on region 12, the third electrode 130 attached on region 13 and the forth electrode 140 attached on region 14).

After the electrode patch 100 attached on the back of the neck BN so as for the electrodes 110-140 to position on regions 11-14, as shown in FIG. 9(b), the patch connector 241 and 242 of the pulsed electrical stimulation neckband 200 is combined with the electrode connector 151 and 152 of the electrode patch 100.

Example Use of a Cervical pTES Apparatus:

1. Connecting the cervical pTES apparatus Butterfly Electrode (this is an example for each of the electrodes provided on the patch body) to the cervical pTES apparatus

Removing the clear backings from the four electrode active areas, then connecting the electrode to the cervical pTES apparatus neckband device. It is necessary to ensure that both electrode snaps are properly seated in the two magnetic receptacles.

2. Placing the electrode patch on the neck with the electrodes correctly positioned

Placing the pulsed electrical stimulation neckband around the neck so that each of the electrodes is positioned at the middle of the base of the neck.

It may be necessary to press the electrode patch down to ensure adherence to the skin.

Electrode positioning (FIG. 9): A simple way to position the electrode in the midline of the neck is to place your finger at the ‘valley’ of the curved shape and feel the bones of the spine. The spacing of the electrodes ensures that current is delivered to a wide area of the cervical plexus and permits robustness to precise placement of the electrode vertically on the neck, a key usability feature given that users are placing the electrode patch themselves without visual feedback.

3. Turning the cervical pTES apparatus on, select a waveform, and begin stimulation.

Pressing the Power/Play button to turn the device on. The LED will turn on.

Then press the Waveform Select button to select the appropriate waveform. The second (green) and third (blue) waveforms performed strongly in physiology assessments.

Starting stimulation by pressing the Power/Play button again

Holding the down intensity button for ˜1 second to go to minimum intensity.

Next, selecting an intensity with the Up Intensity and Down Intensity buttons so that mild skin sensations are felt on the neck.

Stronger stimulation above that which causes a mild skin sensation will not necessarily increase the effectiveness of cervical modulation. The strongest physiological response of relaxation (i.e. suppression of sympathetic activity) generally occurs at a ‘just right’ intensity, and increasing the intensity above this level may reduce the relaxing effects due to activation of pain and/or neuromuscular pathways.

After a stimulation session of generally 10-20 minutes, press the Power/Play button to ramp down stimulation before removing the cervical pTES apparatus and electrode from the neck.

Validation of Acute Cervical pTES Apparatus Performance

In order to begin evaluating the efficacy of an exemplar cervical pTES system to influence sleep onset and the quality of sleep, we tested the acute effects of a cervical pTES apparatus on known biomarkers of physiological relaxation. Here, we were primarily interested in evaluating the ability of the cervical pTES apparatus to stimulate physiological relaxation in an acute setting. We conducted these basic physiological tests using heart rate (HR), measures of heart rate variability (HRV), and facial imaging of near infrared signals as previously described.

Our findings illustrate that pulsed transdermal electrical stimulation (pTES) delivered from cervical pTES apparatus to the cervical plexus is effective at stimulating acute physiological relaxation as described below.

The cervical plexus and its associated circuitry are tightly coupled to the autonomic nervous system and vagal networks, so we first examined whether a cervical pTES apparatus was capable of modulating cardiovascular dynamics. We evaluated the influence of cervical pTES apparatus treatment protocols on heart rate compared to sham treatments. (Sham treatments included both no stimulation control sessions and a 30 second-stimulation protocol at the onset of the treatment period.)

### Experimental Protocol, Heart Rate, and HRV

The basic experimental protocol included a 5 min baseline period, followed by a 10 min sham or pTES treatment session, and finally followed by a 5 min recovery period. Subjects were asked to stare at a fixation spot on the wall or a monitor and sit motionless, staring straight ahead throughout the 20 min testing period. In this first set of experiments, continuous heart rate was recorded using a Polar H7 heart rate monitor and streamed via Bluetooth to a mobile data acquisition system. Data were processed offline to calculate RR intervals and HRV metrics including AVNN, SDNN, pNN50, and the spectral characteristics of HRV (LF, HF, and LF:HF). HR and HRV data were analyzed using paired one-tailed t-tests against baseline values in a within treatment manner. Additionally, raw HR data were transformed to and analyzed as Z-scores using the mean and standard deviation of HR calculated for the baseline period. Unless indicated otherwise, data are shown as mean±SEM.

“LF” is the abbreviation for Low Frequency (it is related to sympathetic nerve) which is related to a physical fatigue and a loss of energy in body. “HF” is the abbreviation for High Frequency (it is related to parasympathetic nerve) which is related to a mental fatigue and stress.

“AVNN” is Average beat-to-beat interval. “SDNN” is Standard Deviation of N-N interval (HRV). “pNN50” is a rate of NN50 (the number of consecutive N-N intervals showing longer difference than 50 ms) to total N-N intervals.

“SEM” is the abbreviation for Standard Error of Mean.

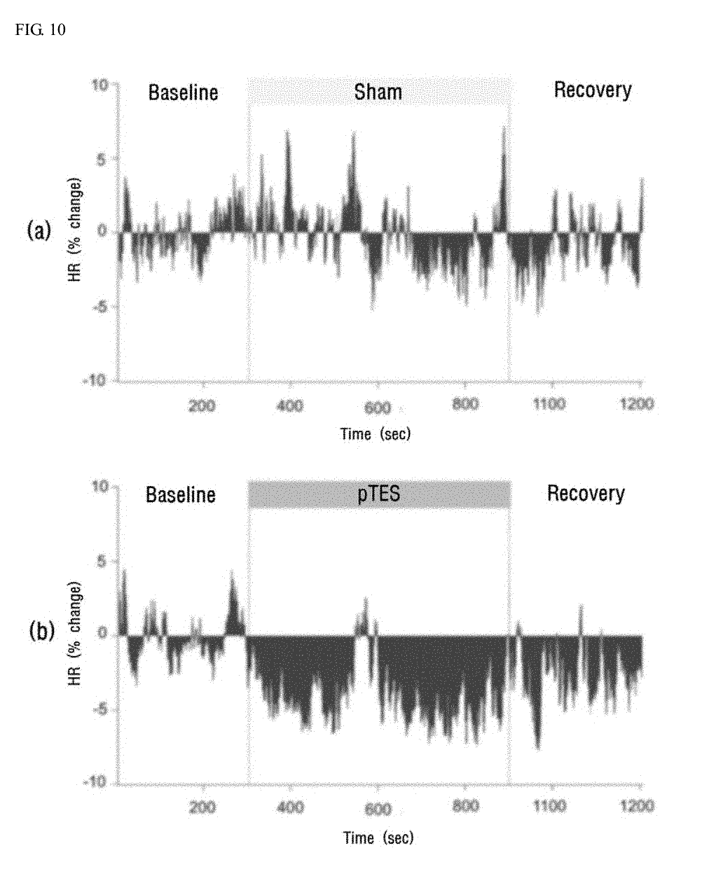

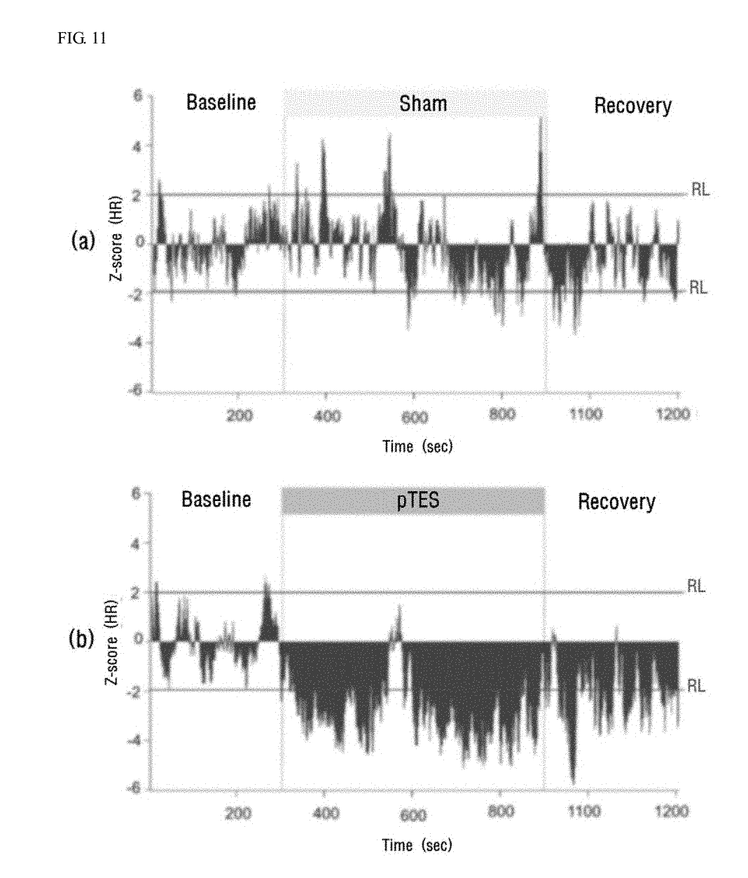

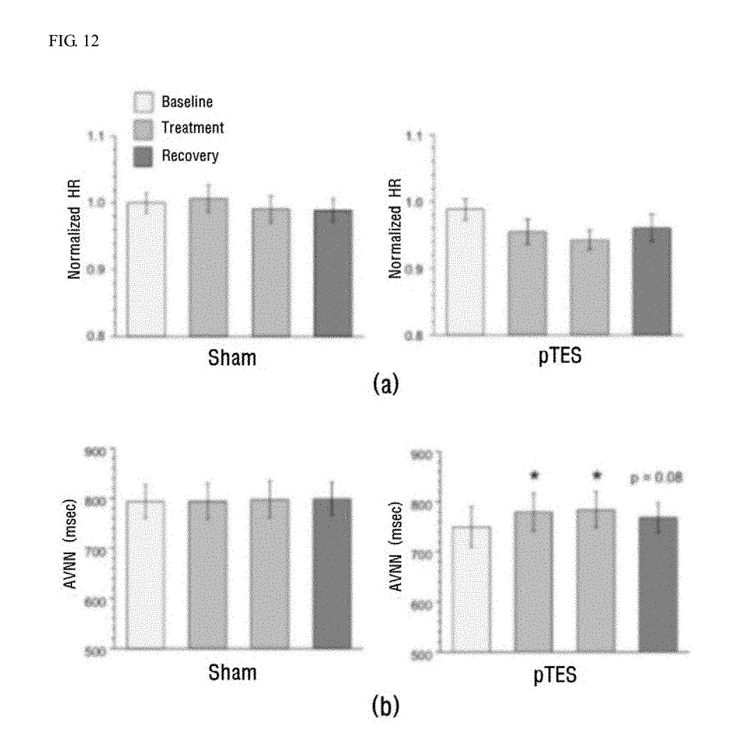

As predicted, we observed that 350 Hz pTES (waveforms 1 and 2) produced significant decreases in HR compared to baseline, whereas sham treatments failed to do so (FIG. 12; n=16; 8 sham, 8 pTES). The mean HR for the control treatment group during the 5 min baseline period (77.15±3.19 bpm) did not significantly change during the sham treatment periods (first 5 min=77.61±3.20 bpm, p=0.22; second 5 min=76.53±3.64 bpm, p=0.29) or 5 min recovery period (76.27±3.25 bpm, p=0.22). As mentioned previously and as shown in FIGS. 10 and 11, pTES treatments did produce significant decreases in mean HR values compared to the 5 min baseline period. The mean 5 min baseline HR for the pTES treatment group (83.16±4.11) dropped significantly during the pTES treatment periods (first 5 min=79.52±3.84 bpm, p=0.01; second 5 min=78.87±3.43 bpm, p=0.003), as well as during the 5 min recovery period (80.01±3.04 bpm, p=0.04). These data are illustrated as baseline normalized values for side-by-side comparisons between control treatments and pTES in FIGS. 10 and 11. Taken together, these data show that pTES produces a significant effect on the autonomic nervous system by stimulating physiological relaxation as observed through a decrease in heart rate (FIGS. 10, 11 and 12).

FIGS. 10 and 11 show that pulsed transdermal electrical stimulation significantly decreases heart rate. FIG. 10 plots illustrate the percent change in heart rate from baseline for sham (FIG. 10(a)) and 350 Hz pTES treatment (FIG. 10(b)). FIG. 11 plots illustrate HR transformed as Z-scores for sham (FIG. 11(a)) and pTES treatment (FIG. 11(b)) and highlight the significant changes from baseline where HR Z-scores cross the changing limit line (RL) of a |Z|=1.96 (p=0.05).

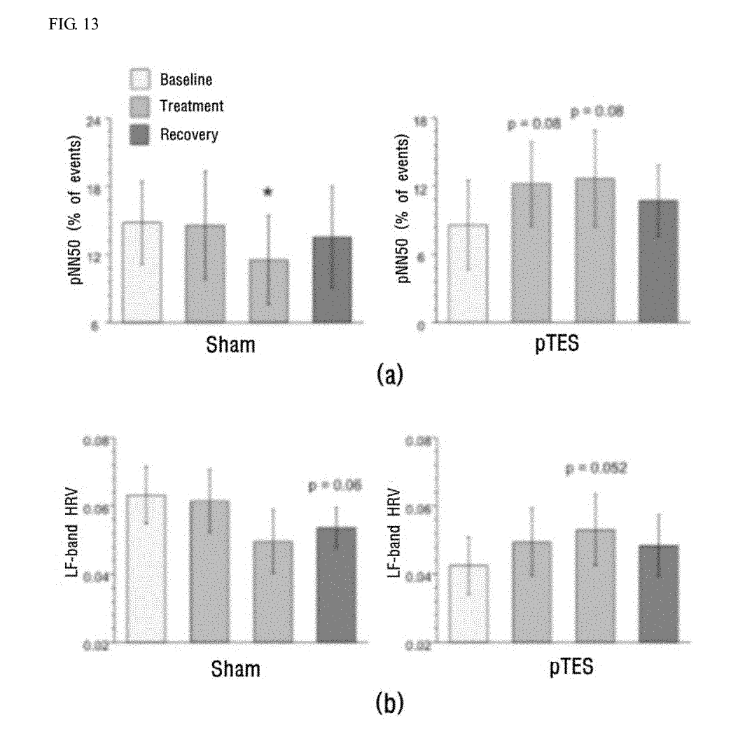

As described above, HRV comparisons were made against baselines in a within-treatment group manner to account for any inter-person variability with respect to baseline HRV values. The between subject (or inter-person) variability contaminates comparisons across treatment groups. Thus, either baseline comparisons as we have performed here or within-subjects, randomized cross-over designs would need to be implemented. The latter was beyond the scope of our proof of concept testing since we are accustomed to the relatively modest strength of effects bestowed by thousands of pTES waveforms in an equally large population of individuals. Interestingly, the magnitude of effects on HR when delivering 350 Hz pTES to the cervical plexus were larger than previously reported findings when pTES was delivered to the trigeminal nerve and the cervical nerves at higher frequencies (a range of 3 kHz and 20 kHz) using the Thync™ device. Similar to the above preliminary findings, we observed larger effects of 350 Hz pTES delivered to the cervical plexus on HRV metrics, compared to previously reported effects of higher frequency pTES delivered to both the trigeminal and cervical nerves. Compared to baseline, 350 Hz pTES produced significant increases in HRV as predicted based on past observations. This was observed as an increase in the average beat-to-beat interval (AVNN), which reflects the decreased heart rate (FIG. 13 and Table 1). There were no significant AVNN changes in response to sham treatments (FIG. 13 and Table 1). The 350 Hz pTES treatments also produced significant increases in the pNN50 and the LF component of HRV compared to baseline measures, whereas sham treatments did not.

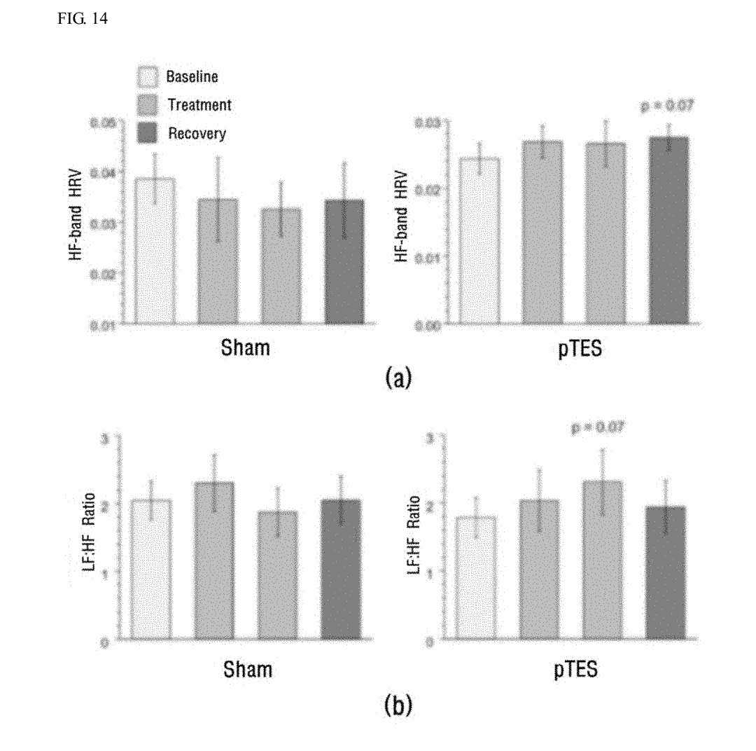

FIGS. 12 to 14 show that pulsed transdermal electrical stimulation of the cervical plexus produces significant changes in autonomic activity as reflected by cardiovascular dynamics. In FIG. 12(a) normalized HR is illustrated by the histograms for both sham (left) and pTES (right) treatment conditions during the 5 min baseline period, the 10 min treatment period, and the 5 min recovery period. As illustrated pTES treatment significantly decreased HR compared to baseline conditions, whereas sham treatments did not. In FIG. 12(b) to FIG. 14(b), data from several HRV metrics are illustrated in the histograms for AVNN, pNN50, LF-band HRV, HF-band HRV, and LF:HF ratio. Sham treatments produced one significant effect on pNN50 that went in the opposite direction of a marginal effect produced by pTES (FIG. 13(a)). Otherwise sham did not produce any significant changes in HRV metrics, but produced some marginal effects on the LF (FIG. 13(b)) bands of HRV that also moved in the opposite direction of pTES treatments compared to baseline. There was a significant increase in AVNN produced by pTES (FIG. 12(b)), which was associated with marginal effects on pNN50 (FIG. 13(a)) and the LF-band (FIG. 13(b)), the HF-band (FIG. 14(a)) and the LF-to-HF ratio (FIG. 14(b)) of HRV. *p≤0.05. p-values <0.10 are indicated by text. See Tables 1-7 for additional details.

TABLE 1
Mean AVNN Values for sham and pTES treatments.

| | | Sham | | | pTES | | |
|---|---|---|---|---|---|---|---|
| | | Mean | | | Mean | | |
| Time | | (msec) | SEM | p-value | (msec) | SEM | p-value |
| Base- | 0-5 min | 793.46 | 32.95 | | 749.52 | 40.08 | |
| line | | | | | | | |
| Treat- | 5-10 min | 794.17 | 35.56 | 0.9431 | 778.69 | 37.39 | 0.0098* |
| ment | | | | | | | |
| Treat- | 10-15 min | 797.63 | 37.05 | 0.7199 | 783.86 | 36.10 | 0.0014* |
| ment | | | | | | | |
| Re- | 15-20 min | 799.07 | 32.79 | 0.6281 | 768.17 | 30.00 | 0.0932 |
| covery | | | | | | | |

We did not observe significant effects on the HRV metric SDNN (Table 2; histograms not shown).

TABLE 2
Mean SDNN Values for sham and pTES treatments.

| | | Sham | | | pTES | | |
|---|---|---|---|---|---|---|---|
| | | Mean | | | Mean | | |
| Time | | (msec) | SEM | p-value | (msec) | SEM | p-value |
| Base- | 0-5 min | 55.78 | 5.83 | | 45.90 | 7.91 | |
| line | | | | | | | |
| Treat- | 5-10 min | 52.88 | 5.25 | 0.6595 | 50.58 | 6.13 | 0.0984 |
| ment | | | | | | | |
| Treat- | 10-15 min | 48.57 | 4.99 | 0.1252 | 50.34 | 6.06 | 0.1385 |
| ment | | | | | | | |
| Re- | 15-20 min | 51.70 | 4.61 | 0.5117 | 51.05 | 6.63 | 0.1574 |
| covery | | | | | | | |

We found that pTES treatments significantly increased the HRV metric rMSSD in the 10-15 min treatment period, while producing marginal increases during both the 5-10 minute treatment period and the recovery treatment period (Table 3; histograms not shown). Sham treatments on the other hand produced a significant decrease in the rMSSD metric during the 10-15 minute treatment period compared to baseline (Table 3; histograms not shown).

TABLE 3
Mean rMSSD Values for sham and pTES treatments.

| | | Sham | | | pTES | | |
|---|---|---|---|---|---|---|---|
| | | Mean | | | Mean | | |
| Time | | (msec) | SEM | p-value | (msec) | SEM | p-value |
| Base- | 0-5 min | 40.16 | 4.84 | | 27.18 | 4.82 | |
| line | | | | | | | |
| Treat- | 5-10 min | 35.95 | 6.27 | 0.2073 | 32.06 | 3.84 | 0.0522 |
| ment | | | | | | | |
| Treat- | 10-15 min | 32.49 | 5.06 | *0.014 | 33.38 | 4.93 | *0.039 |
| ment | | | | | | | |
| Re- | 15-20 min | 34.63 | 5.19 | 0.3227 | 31.65 | 3.90 | 0.0817 |
| covery | | | | | | | |

We found that pTES treatments produced a trend towards increased pNN50 HRV values (Table 4), whereas sham treatment significantly decreased pNN50 during the 10-15 min treatment period compared to baseline. This effect was in the opposite direction of the trending effect of pTES on pNN50 observed (Table 3 and FIG. 13(a)).

TABLE 4
Mean pNN50 Values for sham and pTES treatments.

| | | Sham | | | pTES | | |
|---|---|---|---|---|---|---|---|
| | | Mean | | | Mean | | |
| Time | | (%) | SEM | p-value | (%) | SEM | p-value |
| Baseline | 0-5 min | 14.81 | 3.64 | | 8.60 | 3.92 | |
| Treatment | 5-10 min | 14.59 | 4.76 | 0.4674 | 12.21 | 3.71 | 0.0854 |
| Treatment | 10-15 min | 11.53 | 3.90 | *0.024 | 12.69 | 4.23 | 0.0891 |
| Recovery | 15-20 min | 13.51 | 4.48 | 0.3142 | 10.74 | 3.13 | 0.1368 |

We found that pTES treatments and sham produced marginal, but non-significant effects on the LF-power band of HRV compared to baseline that were again in the opposite directions of each other as shown in Table 5 and FIG. 13(b)).

TABLE 5
Mean LF-band of HRV power spectrum for sham and pTES treatments.

| | | Sham | | | pTES | | |
|---|---|---|---|---|---|---|---|
| Time | | Mean | SEM | p-value | Mean | SEM | p-value |
| Base- | 0-5 min | 0.0632 | 0.01 | | 0.0424 | 0.01 | |
| line | | | | | | | |
| Treat- | 5-10 min | 0.0614 | 0.01 | 0.4282 | 0.0494 | 0.01 | 0.1153 |
| ment | | | | | | | |
| Treat- | 10-15 min | 0.0496 | 0.01 | 0.1645 | 0.0530 | 0.01 | 0.0528 |
| ment | | | | | | | |
| Re- | 15-20 min | 0.0536 | 0.01 | 0.0624 | 0.0484 | 0.01 | 0.1452 |
| covery | | | | | | | |

We found that pTES treatments produced a marginal, but non-significant effect on the HF-band of the HRV power spectrum during the recovery period compared to baseline, but not during the treatment period. The sham treatment failed to produce any effects on the HF-band (Table 5 and FIG. 14(a)).

TABLE 6
Mean HF-band of HRV power spectrum for sham and pTES treatments.

| | | Sham | | | pTES | | |
|---|---|---|---|---|---|---|---|
| Time | | Mean | SEM | p-value | Mean | SEM | p-value |
| Base- | 0-5 min | 0.0385 | 0.005 | | 0.0244 | 0.002 | |
| line | | | | | | | |
| Treat- | 5-10 min | 0.0344 | 0.008 | 0.943 | 0.0268 | 0.002 | 0.226 |
| ment | | | | | | | |
| Treat- | 10-15 min | 0.0326 | 0.005 | 0.720 | 0.0265 | 0.003 | 0.293 |
| ment | | | | | | | |
| Re- | 15-20 min | 0.0343 | 0.007 | 0.628 | 0.0275 | 0.002 | 0.075 |
| covery | | | | | | | |

We found that pTES treatments produced a marginal, but non-significant effect on the LF:HF ratio during the 10-15 min stimulation period compared to baseline. The sham treatment failed to produce any effects on the LF:HF ratio (Table 5 and FIG. 14(b)).

TABLE 7
Mean LF:HF ratio of HRV power spectrum
for sham and pTES treatments.

| | | Sham | | | pTES | | |
|---|---|---|---|---|---|---|---|
| Time | | Mean | SEM | p-value | Mean | SEM | p-value |
| Base- | 0-5 min | 2.0461 | 0.282 | | 1.7877 | 0.290 | |
| line | | | | | | | |
| Treat- | 5-10 min | 2.3010 | 0.414 | 0.943 | 2.0360 | 0.451 | 0.159 |
| ment | | | | | | | |
| Treat- | 10-15 min | 1.8736 | 0.356 | 0.720 | 2.3099 | 0.476 | 0.071 |
| ment | | | | | | | |
| Re- | 15-20 min | 2.0490 | 0.350 | 0.628 | 1.9431 | 0.385 | 0.221 |
| covery | | | | | | | |

### FLIR Imaging of Facial Temperature

In a second set of experiments we also evaluated facial temperatures using a forward-looking infrared (FLIR) camera. As previously described, skin temperature reflects sudomotor activity that is regulated by norepinephrine (NE) controlling vasodilation and vasoconstriction, as well as the activity of sweat glands. When NE activity is suppressed there is a resulting increase in skin temperature triggered by vasodilation of capillaries. This is indicative of physiological relaxation, whereas decreased skin temperatures of the face reflect a stressed state.

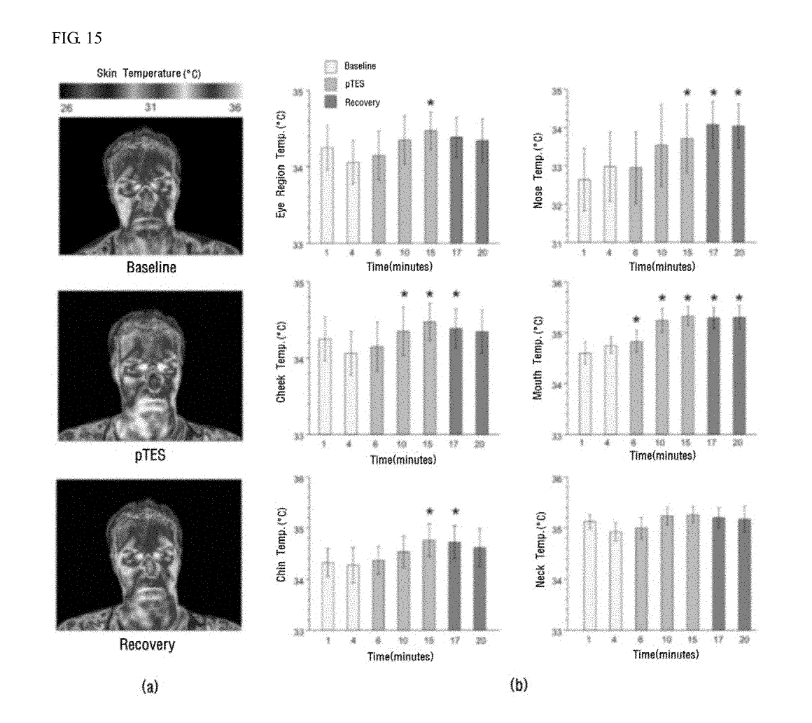

The experimental paradigm was as previously described with a 5 minute baseline, a 10 min pTES treatment period followed by a 5 min recovery period during all of which subjects were asked to attend to a fixation spot on a screen or wall. We found that pTES (n=8) caused a significant increase in skin temperatures versus baseline analyzed in a manner as similarly described (Table 8 and FIG. 15). We did not conduct sham treatments due to our extensive previous experience with this assay and consistent findings in a sham condition of no significant changes in temperature in response to sham pTES.

The changes we observed here in response to 350 Hz cervical pTES (waveforms 1 and 2) are consistent with our previous observations that trigeminal and cervical nerve modulation triggers changes in skin temperatures in a manner that is consistent with the induction of physiological relaxation. Collectively, our observations provide preliminary evidence that cervical plexus modulation with pTES reliably inhibits sympathetic nervous system activity. Taken in context with our previous studies showing that improved sleep and morning mood occur when sympathetic nervous system activity is suppressed before sleep, we hypothesize that cervical plexus pTES will be able to significantly improve sleep quality by reducing sympathetic tone and sympathetic activity.

FIG. 15 shows that pulsed transdermal electrical stimulation significantly increases facial temperature. FIG. 15(a) shows example FLIR images are illustrated for baseline, pTES treatment, and recovery periods. FIG. 15(b) shows histograms of average temperatures for different regions of the face during baseline, pTES treatment, and the recovery period. *p≤0.05.

TABLE 8
Mean temperature values of facial regions in response to 350 Hz cervical pTES
across time.

| | | FACE REGION TEMPERATURE (° C.) | | | | | | |
|---|---|---|---|---|---|---|---|---|
| Time | | Chin | Mouth | Nose | Cheek | Eyes | Forehead | Neck |
| 1 min | Mean | 34.325 | 34.600 | 32.638 | 33.813 | 34.250 | 34.913 | 35.125 |
| | SEM | 0.273 | 0.216 | 0.822 | 0.135 | 0.290 | 0.255 | 0.135 |
| 6 min | Mean | 34.363 | 34.825 | 32.950 | 33.913 | 34.150 | 34.813 | 35.000 |
| | SEM | 0.266 | 0.219 | 0.943 | 0.220 | 0.321 | 0.308 | 0.206 |
| | p-value | 0.398 | 0.247 | 0.283 | 0.210 | 0.212 | 0.200 | 0.129 |
| 10 min | Mean | 34.538 | 35.238 | 33.538 | 34.113 | 34.350 | 34.950 | 35.238 |
| | SEM | 0.303 | 0.235 | 1.075 | 0.205 | 0.318 | 0.285 | 0.176 |
| | p-value | 0.100 | *0.042 | 0.094 | *0.043 | 0.241 | 0.331 | 0.134 |
| 15 min | Mean | 34.763 | 35.325 | 33.713 | 34.313 | 34.475 | 34.025 | 35.263 |
| | SEM | 0.316 | 0.189 | 0.897 | 0.238 | 0.242 | 0.252 | 0.169 |
| | p-value | *0.007 | *0.019 | *0.052 | *0.008 | *0.048 | 0.099 | 0.090 |
| 17 min | Mean | 34.725 | 35.288 | 34.075 | 34.213 | 34.388 | 34.913 | 35.200 |
| | SEM | 0.319 | 0.212 | 0.0614 | 0.210 | 0.258 | 0.278 | 0.195 |
| | p-value | *0.002 | *0.042 | *0.011 | *0.015 | 0.138 | 0.500 | 0.275 |
| 20 min | Mean | 34.614 | 35.300 | 34.043 | 34.143 | 34.343 | 34.886 | 35.171 |
| | SEM | 0.376 | 0.225 | 0.576 | 0.318 | 0.283 | 0.305 | 0.255 |
| | p-value | 0.062 | *0.029 | *0.014 | 0.092 | 0.334 | 0.443 | 0.381 |

While multiple embodiments are disclosed, still other embodiments of the present invention will become apparent to those skilled in the art from this detailed description. The invention is capable of myriad modifications in various obvious aspects, all without departing from the spirit and scope of the present invention. Accordingly, the drawings and descriptions are to be regarded as illustrative in nature and not restrictive.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” (or primary and secondary) may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A pulsed transdermal electrical stimulation apparatus targeting cervical nerves on the back of the neck comprising:
a pulsed electrical stimulation neckband including:
a neckband body configured to be worn on the neck;
a stimulation generator configured to generate signals for pulsed electrical stimulation, wherein the stimulation generator is provided on a location of the back of the neck in the neckband body;
a patch connector provided on the neckband body in connection with the stimulation generator; and
a controller configured to control the stimulation generator, and
an electrode patch including:
a patch body for being attached on the location of the back of the neck;
an electrode connector provided on the patch body and detachably combined with the patch connector so as to electrically connect to the patch connector; and
electrodes for providing the signals for pulsed electrical stimulation of the stimulation generator transferred from the electrode connector to a skin of the back of the neck on which the patch body is attached.

2. The pulsed transdermal electrical stimulation apparatus of claim 1, wherein the patch body forms electrode arranging portions corresponding to four regions to be stimulated targeting cervical nerves on the back of the neck, the electrodes include a first electrode, a second electrode, a third electrode and a forth electrode provided on each of the electrode arranging portions, and
the first electrode and the second electrode make a pair of electrodes, and the third electrode and the forth electrode make another pair of electrodes, wherein each the pair of electrodes provide a pulsed electrical stimulation by a bipolar manner.

3. The pulsed transdermal electrical stimulation apparatus of claim 2, wherein the electrode connector includes a first connector connected with the first electrode and the third electrode for assigning a polarity to the first electrode and the third electrode, and a second connector connected with the second electrode and the forth electrode for assigning the opposite polarity to the second electrode and the forth electrode, the signals for pulsed electrical stimulation of the stimulation generator are transferred to each of the electrodes through the first connector and the second connector, and the pulsed electrical stimulation energies between the first electrode and the second electrode and between the third electrode and the forth electrode are respectively transferred to the four regions to be stimulated targeting cervical nerves on the back of the neck.

4. The pulsed transdermal electrical stimulation apparatus of claim 2, wherein each of the electrodes provides at least one of polar members for forming an electrode providing an electrical stimulation, and the patch body includes a conductive pattern layer coated by conductive material for electrically connecting with the electrode connector and at least one of the polar members in each of the electrodes.

5. The pulsed transdermal electrical stimulation apparatus of claim 4, wherein the each of the electrodes includes a conductive hydrogel layer for sticking to the skin and delivering the electrical stimulation to the regions on the back of the neck through at least one of the polar members.

6. A pulsed transdermal electrical stimulation apparatus targeting cervical nerves on the back of the neck comprising:
a pulsed electrical stimulation neckband configured to deliver pulsed symmetric charge balanced electrical stimulation having a frequency between 350 and 500 Hz, a pulse width of 200 to 250 microseconds, an pulse interval of 125 to 375 microseconds, and a peak amplitude of 0.5 to 10 mA;
an electrode patch configured to connect electromechanically to the pulsed electrical stimulation neckband and provide two or more electrically conductive electrodes to a user's skin; and
a user control for activating the pulsed electrical stimulation neckband to deliver electrical stimulation transdermally to a subject.

7. The pulsed transdermal electrical stimulation apparatus of claim 6, wherein the heart rate of a user decreases in response to electrical stimulation

8. The pulsed transdermal electrical stimulation apparatus of claim 6, wherein the heart rate variability of a user changes in response to electrical stimulation.

9. The pulsed transdermal electrical stimulation apparatus of claim 6, wherein the skin temperature on a user's face increases in response to electrical stimulation.

10. The pulsed transdermal electrical stimulation apparatus of claim 6, wherein the electrode patch has four active areas.

11. The pulsed transdermal electrical stimulation apparatus of claim 10, wherein the active areas of an electrode assembly are round and roughly symmetrically positioned across the midline of the spine on the neck.

12. A control method of a pulsed transdermal electrical stimulation apparatus targeting cervical nerves on the back of the neck, the method comprising:
electromechanically connecting with a pulsed electrical stimulation neckband and an electrode patch as positioning symmetrically on the back of the neck the electrode patch and wearing the pulsed electrical stimulation neckband; and
controlling a stimulation generator of the pulsed electrical stimulation neckband to deliver pulsed symmetric charge balanced electrical stimulation having a frequency between 350 and 500 Hz, a pulse width of 200 to 250 microseconds, an pulse interval of 125 to 375 microseconds, and a peak amplitude of 0.5 to 10 mA so as to transfer the stimulation to cervical nerves on the back of the neck through the electrode patch.
